(19) United States
(12) Patent Application Publication
(10) Pub. No.: US 2008/0317814 A1
(43) Pub. Date: Dec. 25, 2008

(54) Tube for Connecting Marteriovenous and Interposition for Medical Operation

(75) Inventors: Dae-Joong Kim, Seongnam-si (KR); Chul-Soo Gim, Pyeongtaek-si (KR); Jai-Young Ko, Pyeongtaek-si (KR); Jong-Sang Park, Seoul (KR); Tae-Gun Kwon, Seoul (KR); Byung-Ha Lee, Gunpo-si (KR); Woo-Kyung Lee, Paju-si (KR); Hye-Yeong Nam, Cheongju-si (KR); Hyun-Jung Lim, Seoul (KR)

Correspondence Address: Jefferson IP Law, LLP, 1730 M Street, NW, Suite 807, Washington, DC, 20036, US

(73) Assignee: ACCESS PLUS CO., LTD., Seoul (KR)

(21) Appl. No.: 12/094,022

(22) PCT Filed: Nov. 17, 2005
(86) PCT No.: PCT/KR2005/003906

§ 371 Date: May 16, 2008

(51) Int. Cl. A61F 2/06

(52) U.S. Cl. 424/423

(57) ABSTRACT

Disclosed herein are a tube for arteriovenous anastomosis useful in hemodialysis patients and an insert for use in medicinal operation in the body. The tube or the insert are repetitively treated with a medicament on the surface thereof, which provides stable communication between an artery and a vein of a patient under hemodialysis and can greatly reduce stenosis at arteriovenous connections by releasing the medicament at a suitable rate over a prolonged period of time.

### TECHNICAL FIELD

The present invention relates to a tube for arteriovenous anastomosis. More particularly, the present invention relates to a technology which can provide stable communication between the artery and the vein of a patient under hemodialysis and can greatly reduce stenosis at arteriovenous connections.

### BACKGROUND ART

Hemodialysis, a method for treating acute or chronic renal failure, is periodically applied to patients suffering from severe renal failure. Recently, the significance of hemodialysis has increased with the increasing number of patients with renal failure.

For most hemodialysis patients, diabetic mellitus or hypertension is found to be a basic cause, entailing serious arteriosclerosis. Successful hemodialysis requires the elimination of factors interrupting blood flow at arteriovenous connections. Intensive studies have been conducted on such hemodialysis topics.

Meanwhile, an artificial vessel (or a tube for arteriovenous anastomosis) has been developed as an alternative for guiding blood flow to compensate for the stenosis or significant dysfunction of real blood vessels. Depending on chemical compositions and physical properties, including porosity, elasticity, elasticity, surface structure, etc., artificial blood vessels vary in patency.

Usually, structures which can be used as tubes for arteriovenous anastomosis are made from e-PTFE (expanded polytetrafluoroethylene). A microporous thin film made by multi-axially drawing e-PTFE at high temperature and high pressure has such a low friction coefficient as to show antithrombogenicity, e.g., not to allow proteins to adhere to the surface thereof when it is in contact with blood. These properties allow the film to be applied to the tube structures.

Over autogenous arteriovenous fistula, tubes for arteriovenous anastomosis have advantages in performing hemodialysis in patients. However, angiostenosis may occur at connection between the artificial graft and arteriovenous vessels, interrupting blood flow. Accordingly, the tube for arteriovenous anastomosis is required to be transplanted again in order to conduct hemodialysis.

Studies for solving the angiostenosis problem have been conducted, leading to the finding that the overgrowth of endothelial cells of the vessel is a cause of the angiostenosis occurring at the connection between the tube for arteriovenous anastomosis and the vessel. The present inventor developed a novel hemodialysis tube in a previous study of the present inventor and filed an application for a patent on the tube in international patent application No. PCT/KR2005/001633, titled “Hemodialysis Tube Treated with Medicament on Surface thereof for Connecting Artery to Vein” (hereinafter referred to as “the prior art”).

The prior art pertains to a technique for surface-treating (coating) at least both opposing ends of the tube structure with a medicament for inhibiting the overgrowth of endothelial cells of vessels. According to the prior art, the medicament is released from the connections between the tube arteriovenous anastomosis and the vessel to inhibit endothelial cells of the vessel from growing excessively, thereby preventing angiostenosis thereat.

In practice, after being transplanted, the tube structure according to the prior art shows a significant effect of suppressing the overgrowth of endothelial cells. However, this suppressive effect varies depending on the manner in which the medicament applied to the surface of the transplanted tube for arteriovenous anastomosis is released. In many cases, the surface-treated medicament is released in a large amount in an early stage so that the beneficial effect of the medicament does not last.

### DISCLOSURE OF INVENTION

### Technical Problem

Accordingly, the present invention has been made keeping in mind the above problems occurring in the prior art, and an object of the present invention is to provide a tube for arteriovenous anastomosis which can release a medicament layered thereon persistently at a suitable rate over a prolonged period of time, and a method for preparing the same.

### Technical Solution

In accordance with the present invention, the above object can be accomplished by the provision of a method for preparing a tube for arteriovenous anastomosis, comprising: a primary surface treatment step comprising: dissolving a medicament at a predetermined concentration in a solvent, the medicament being able to inhibit the overgrowth of blood vessel endothelial cells; immersing the entire portion or opposite end portions of a tube structure for a predetermined period of time; drawing the tube structure out of the solution; and drying the tube structure; and a secondary surface treatment step comprising: immersing the entire portion or opposite end portions of the primarily surface-treated tube structure in a solution of a predetermined concentration of a medicament in a solvent or in a solution having no medicament for a predetermined period of time; drawing the tube structure out of the solution; and drying the tube structure.

In a modification, the second surface treatment step is repeated at least once more.

Preferably, the time period for which the tube structure is immersed in the secondary surface treatment step is shorter than that for which the tube structure is immersed in the primary surface treatment step.

In accordance with another aspect of the present invention, a method is provided for preparing a tube for arteriovenous anastomosis, in which a medicament capable of inhibiting the overgrowth of blood vessel endothelial cells is layered on a tube structure, followed by eliminating the portion of the medicament that is not firmly adhered to the tube structure.

In a modification of this aspect, a surface treatment of the tube structure with a medicament capable of inhibiting the overgrowth of blood vessel endothelial cells is further carried out after the elimination.

In accordance with a further aspect of the present invention, a method is provided for preparing an insert for use in medicinal operation in the body, which comprises: a primary surface treatment step comprising: dissolving a medicament at a predetermined concentration in a solvent, the medicament being able to inhibit the overgrowth of blood vessel endothelial cells; immersing the entire portion or opposite end portions of a structure for a predetermined period of time; drawing the structure out of the solution; and drying the structure; and a secondary surface treatment step comprising: immersing the entire portion or opposite end portions of the primarily surface-treated structure in a solution of a predetermined concentration of a medicament in a solvent or in a solution having no medicament for a predetermined period of time; drawing the structure out of the solution; and drying the structure.

In a modification of this aspect, second surface treatment step is repeated at least once more.

Preferably, the time period for which the structure is immersed in the secondary surface treatment step is shorter than that for which the structure is immersed in the primary surface treatment step.

In accordance with still a further aspect of the present invention, a method is provided for preparing an insert for use in medicinal operation in the body, in which a medicament capable of inhibiting the overgrowth of blood vessel endothelial cells is layered on a structure, followed by eliminating the portion of the medicament which is not firmly adhered to the structure.

In a modification of this aspect, a surface treatment of the structure with a medicament capable of inhibiting the overgrowth of blood vessel endothelial cells is further carried out after the elimination.

### ADVANTAGEOUS EFFECTS

When the tube for arteriovenous anastomosis according to the present invention is inserted into the body so as to conduct hemodialysis, it assures stable hemodialysis because the medicament applied on the tube is released persistently over a prolonged period of time. Also, an insert for use in medicinal operation in the body, prepared according to the present invention, exhibits the same drug release behavior.

### BRIEF DESCRIPTION OF THE DRAWINGS

FIG. 1 is a flow chart illustrating the preparation of a tube for arteriovenous anastomosis in accordance with an embodiment of the present invention; and

FIG. 2 is a partially broken perspective view showing the tube prepared according to the method illustrated in FIG. 1.

### BEST MODE FOR CARRYING OUT THE INVENTION

Reference should now be made to the drawings, in which the same reference numerals are used throughout the different drawings to designate the same or similar components.

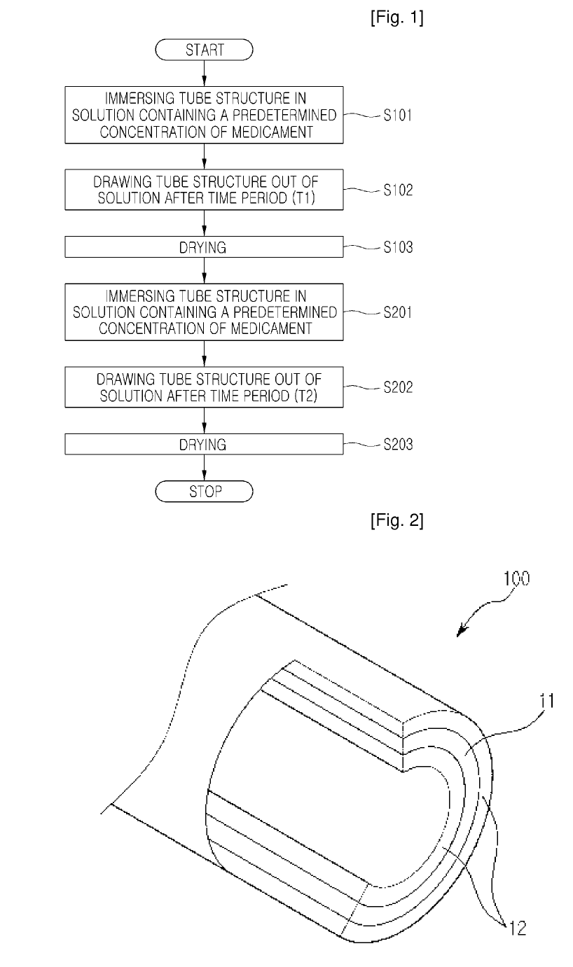

FIG. 1 illustrates a method for preparing a tube for arteriovenous anastomosis in accordance with the present invention, and the structure of the tube is rather exaggeratedly shown in a partially broken perspective view in FIG. 2.

Ahead of a method for preparing a tube for arteriovenous anastomosis in accordance with an embodiment, materials for the tube will be explained. A tube structure, medicaments, and solvents are needed for the preparation of the tube. A medicament or solvent used in one surface treatment step may be the same as or different from that used in another step.

The tube structure has a roughly cylindrical form and is made from a porous e-PTFE film, which is obtained by drawing PTFE in multiple directions at a high temperature under a high pressure. Preferably, Gore-Tex is extruded into a cylindrical form.

To be applied to the surface of the tube structure, a medicament is required to have inhibitory activity against the overgrowth of endothelial cells of vessels, as well as no side effects. Preferably, Paclitaxel or Rapamycin may be used as a medicament for treating the tube structure. Medicaments used in plural surface treatment processes, as mentioned above, may be the same or different.

As for the solvent, it dissolves the medicament so as to treat the surface of the tube structure with it. Most suitable are organic solvents which not only can dissolve the medicament, such as Paclitaxel or Rapamycin, but also cause no problems, with acetone being preferred. Also, solvents used in plural surface treatment processes, as mentioned above, may be the same or different.

After material preparation, a tube for arteriovenous anastomosis is prepared according to the process shown in the flow chart of FIG. 1.

1. Primary Surface Treatment

A medicament is dissolved at a suitable concentration (C1) in a solvent (S1).

A tube structure is immersed in a solution of the medicament in the solvent. In this connection, the tube structure may be treated with the solution on the entire surface thereof or on a partial surface of opposite ends that will be junctions with the artery and the vein, respectively.

After immersion for a predetermined time period (T1), the primary surface treatment is completed by drawing the tube structure out of the solution (S102) and drying it (S103).

2. Secondary Surface Treatment

The primarily surface-treated tube structure is immersed in a solvent (S2) containing a predetermined concentration (C2) of a medicament (S201). During the immersion, the medicament already applied on the tube structure in the primary surface treatment is dissolved into the solvent (S2) if it is not firmly attached, or is overlaid with fresh medicament if it is firmly attached.

After the immersion of the primarily surface-treated tube structure for a predetermined time period (T2), the second surface treatment is completed by drawing the tube structure out of the solution (S202) and drying it (S203).

At least one repetition of the second surface treatment assures that only the medicaments which are firmly attached to the tube structure remain on the tube structure and are covered with a fresh one or another medicament, while those that are not firmly attached thereonto are eliminated from the tube structure. Ultimately, the tube structure has medicaments firmly attached to the surface thereof.

The medicaments or solvents used in the primary and the secondary surface treatment, as mentioned above, may be the same or different.

The immersion time period (T2) in the secondary surface treatment may be shorter than that (T1) in the primary surface treatment. In this study, desired results are obtained when the time period (T2) is significantly shorter than the time period (T1), but there remains a subject of setting the best relationship therebetween.

Alternatively, the secondary surface treatment may comprise immersing the primarily surface-treated tube structure in a solvent containing no medicaments so as to dissolve readily releasable medicament into the solvent. In this case, the medicament may remain very firmly adhered to the tube structure, so that it is prevented from being excessively released in an early stage.

When the tube for arteriovenous anastomosis is prepared in this process, absolute amounts of the medicament released in an early or late stage may be reduced, but this is found to be compensatable by increasing the concentration of the medicament upon the primary surface treatment.

With reference to FIG. 2, a tube for arteriovenous anastomosis 100, prepared in the process described above, is shown in a partially broken perspective view.

As seen in this figure, the tube for arteriovenous anastomosis 100 comprises a generally cylindrical tube structure 11 and a coating layer 12 applied to both an inner and an outer surface of the tube structure 11.

When being inserted into the body, the tube for arteriovenous anastomosis 100 allows the medicament to be released slowly and persistently over a prolonged period of time because the medicament of the coating layer 12 is so firmly adhered to the tube structure 11 as not to be easily dissolved out.

Although the present invention is explained with a tube for arteriovenous anastomosis in accordance with an embodiment, it must be noted that the surface treatment of the present invention can be applied to inserts which are required to interrupt the overgrowth of blood vessel endothelial cells as grafts in the body.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described hereinbefore, the tube for arteriovenous anastomosis according to the present invention can stably connect an artery to a vein therethrough in hemodialysis patients, with an improvement in blood vessel blockage. Accordingly, the present invention is very useful for patients who are forced to undergo periodic hemodialysis.

1. A method for preparing a tube for arteriovenous anastomosis, comprising:
a primary surface treatment step comprising:
dissolving a medicament at a predetermined concentration in a solvent, the medicament being able to inhibit the overgrowth of blood vessel endothelial cells;
immersing the entire portion or opposite end portions of a tube structure for a predetermined period of time;
drawing the tube structure out of the solution; and
drying the tube structure; and
a secondary surface treatment step comprising:
immersing the entire portion or opposite end portions of the primarily surface-treated tube structure in a solution of a predetermined concentration of a medicament in a solvent or in a solution having no medicament for a predetermined period of time;
drawing the tube structure out of the solution; and
drying the tube structure.

2. The method as defined in claim 1, wherein the second surface treatment step is repeated at least once more.

3. The method as defined in claim 1, wherein the time period for which the tube structure is immersed in the secondary surface treatment step is shorter than that for which the tube structure is immersed in the primary surface treatment step.

4. The method as set forth in claim 1, wherein the solvent used in the primary or the secondary surface treatment step is an organic solvent including acetone.

5. A method for preparing a tube for arteriovenous anastomosis, in which a medicament capable of inhibiting the overgrowth of blood vessel endothelial cells is layered on a tube structure, followed by eliminating a portion of the medicament that is not firmly adhered to the tube structure.

6. The method as defined in claim 5, in which a surface treatment of the tube structure with a medicament capable of inhibiting the overgrowth of blood vessel endothelial cells is further carried out after the elimination.

7. A method for preparing an insert for use in medicinal operation in the body, comprising:
a primary surface treatment step comprising:
dissolving a medicament at a predetermined concentration in a solvent, the medicament being able to inhibit the overgrowth of blood vessel endothelial cells;
immersing the entire portion or opposite end portions of a structure for a predetermined period of time;
drawing the structure out of the solution; and
drying the structure; and
a secondary surface treatment step comprising:
immersing the entire portion or opposite end portions of the primarily surface-treated structure in a solution of a predetermined concentration of a medicament in a solvent or in a solution having no medicament for a predetermined period of time;
drawing the structure out of the solution; and
drying the structure.

8. The method as defined in claim 7, wherein the second surface treatment step is repeated at least once more.

9. The method as defined in claim 7, wherein the time period for which the structure is immersed in the secondary surface treatment step is shorter than that for which the structure is immersed in the primary surface treatment step.

10. The method as set forth in claim 7, wherein the solvent used in the primary or the secondary surface treatment step is an organic solvent including acetone.

11. A method for preparing an insert for use in medicinal operation in the body, in which a medicament capable of inhibiting the overgrowth of blood vessel endothelial cells is layered on a structure, followed by eliminating a portion of the medicament that is not firmly adhered to the structure.

12. The method as defined in claim 11, in which a surface treatment of the structure with a medicament capable of inhibiting the overgrowth of blood vessel endothelial cells is further carried out after the elimination.
